(19) United States
(12) Patent Application Publication
(10) Pub. No.: US 2025/0027795 A1
(43) Pub. Date: Jan. 23, 2025

(54) Systems and Methods to Integrate Environmental Information into Measurement Metadata in an Electronic Laboratory Notebook Environment

(71) Applicant: Elemental Machines, Inc., Cambridge, MA (US)

(72) Inventors: Ian Harding, Wells (GB); Sridhar Iyengar, Salem, NH (US)

(73) Assignee: Elemental Machines, Inc., Cambridge, MA (US)

(21) Appl. No.: 18/796,018

(22) Filed: Aug. 6, 2024

Related U.S. Application Data
(63) Continuation of application No. 16/589,347, filed on Oct. 1, 2019, now abandoned.
(60) Provisional application No. 62/739,427, filed on Oct. 1, 2018.

(51) Int. Cl. G01D 9/02; G06F 16/93; G06F 30/20; G06F 111/10

(52) U.S. Cl. CPC G01D 9/02; G06F 16/93; G06F 30/20; G06F 2111/10

(57) ABSTRACT

An empirical data management system (EDMS), such as an electronic laboratory notebook (ELN) system, includes an application server running an EDMS server application, a data storage system containing data in communication with the application server, and an environmental sensor unit in communication with the application server. The data comprises environmental data received from the environmental sensor unit.

### CROSS-REFERENCE TO RELATED APPLICATIONS AND PRIORITY

This application is related to and claims the benefit of U.S. Prov. Application Ser. No. 62/739,427 filed on Oct. 1, 2018 which is incorporated in its entirety herein by reference for all purposes.

### BACKGROUND OF THE INVENTION

Primary documentation of information has typically been in the form of notebooks (for example, laboratory notebooks, financial transaction ledgers, etc.), which have evolved over time to support legal, supply-chain, manufacturing, healthcare, financial and intellectual-property activities as well as scientific activities. However, with computerization of organizations (such as laboratories, factories, hospitals, etc.), data is now commonly collected and stored in electronic form, analyzed in electronic form and published in electronic form, rendering hand-written notebooks an increasingly anachronistic method of primary record keeping.

One example is with how information and data are collected in scientific applications and processes. Looking at researchers and scientists in particular, the persistence of hand-written laboratory notebooks is not simply a reflection of conservatism on behalf of scientists, but is in part attributable to legal requirements, with counter-signed, dated notebook entries being a simple way to demonstrate for intellectual property purposes the time of invention and for compliance with Good Laboratory Practice (GLP) regulations that the record is original and unaltered. However, electronic data collection and storage systems are now becoming available that offer compliance with these regulations.

Systems for electronic data collection and storage (e.g. an empirical data management system (EDMS)) can be divided into at least two types. A first type is the Laboratory Information Management System (LIMS), a software system dedicated to managing laboratory-based information such as sensor monitoring, workflow and sample tracking, and collecting the data these generate in an environment that complies with GLP principles for electronic data. The typical customers for LIMS are laboratory managers. LIMSs provide a centralized data repository that complies with a range of regulations for electronic storage and support various methods of using the data, such as alerts and monitoring, a GUI dashboard etc. Example LIMS include those sold under the tradenames TetraScience (by TetraScience), DeviceLink and Smart Vue (both by Thermo Fisher Scientific), Tiamo (by Metrohm), Monnit (by Monnit), Rees (by ReesScientific), SmartSense (by Digi), Minus80 (by Minus80monitoring), Tempurity (by Networked Robotics), VisioNize (by Eppendorf), Traxx (by Klatu) and Model AMS (by Hampshire Controls Corp).

A second type of an EDMS system is the Scientific Data Management System (SDMS). More ambitious in scope than a LIMS, an SDMS collects and manages data from larger scientific instruments, providing fully compliant data storage, various management functions for example workflow management, equipment management (scheduling use and maintenance) and an Electronic Laboratory Notebook (ELN). ELNs provide to users an interface to the system that allows them to capture, manage, securely share, and permanently archive and retrieve electronic records in ways that provide the same legal, regulatory, technical and scientific compliance that is provided to the source data. This ELN interface provides context and structure to different types of data; a generic form of ELN gives a flexible platform to support research work, embedding images, sound files, representations of data from a range of instruments and analysis packages into a narrative contained in descriptive text, while more specific applications provide more structured interfaces tailored to particular tasks. The generic form can thus provide validation of ‘first to invent’ during the patent process and a platform to share work across a group, while more specific applications can be tailored to provide compliance with individual GLP requirements and records destined for archiving. The typical customers for SDMS's are researchers. Example SDMSs include those sold under the tradenames StarLims (by Abbott Informatics), Core (by Thermo Fisher Scientific), LabInspector (by Stack Wave), LogiLab (by Agaram Technologies), NuGenesis (by Waters), sciCloud (by LabLynx) and Scilligence SDMS (by Scilligence).

Instrument data can be embedded in ELN entries according to methods disclosed in US patent application Ser. No. 2007/0208800 and U.S. Pat. Nos. 8,984,083, 8,548,950 etc. When data is embedded it is common that only part of data is immediately visible in the ELN, with contextual data known as ‘metadata’ being associated with the embedded data but not immediately visible. FIG. 1 gives examples of file structures to support metadata, but less hierarchical structure is also possible, such as described in U.S. Pat. No. 9,954,976. ELNs commonly give ready access to metadata, which gives ELNs an advantage over traditional laboratory notebooks where associated data is either entered into the notebook manually or not at all, potentially leading to a situation where ELNs offer much richer contextual data than the traditional laboratory notebook. This is because in addition to manual entry, ELNs can collect metadata automatically.

Metadata is attached to data, often in a file hierarchy, as data is moved from the instrument that generated it, through the file (e.g. database) where it is stored, through any analytical packages that, or modules configured with logic to, manipulate the data and to the interfaces where it is used such as ELNs. One ordinarily skilled in the art therefore recognizes that metadata can contain various forms of information that reflect this movement. A first type of metadata is that associated with the measuring instrument that generated the data: U.S. Pat. No. 9,489,485 describes this as data that gives meaning and context to the interpretation of the measurements; U.S. Pat. No. 9,954,976 describes instrument GUI display data as a type of metadata. Such metadata can cover not only instrument settings, but also make, model and serial number of measuring equipment, an institution's asset number and/or identity number within a quality system, personnel running the instrument etc., with this type of metadata being appended to the measurement data as it is generated by the instrument or passes through the control unit associated with the instrument (e.g. a PC or module programmed with logic used in its operation). A second type of metadata is that associated with the Local Area Network (LAN) through which the data passes, such as timestamps of recording and system topology, and the data's position in an information hierarchy, such as research group, project, grant, experiment, sample etc. U.S. Pat. No. 7,555,492 describes a series of such annotations after measurement data: tube and reagent information, sample information, subject information and study and experiment information. A third type of metadata is that appended by scientists. Example methods to support manual identification of data to be appended are described in U.S. Pat. Nos. 8,984,083 and 9,489,485.

Research into metadata appended by scientists is reported in ‘Creating Context for the Experiment Record. User-Defined Metadata: Investigations into Metadata Usage in the LabTrove ELN’ by C. Willoughby, C. L. Bird, S. J. Coles and J. G. Frey in the Journal of Chemical Information and Modeling, 2014, Vol 54 pp 3268-3283. This study shows that what scientists think to add to the metadata follows what has already been established; ELN sections are given finer granularity and tailored terms that describe how those sections fit into alternative hierarchies (in terms used in the study, these are ‘high-level’ classifications, both ‘things’ and less frequently ‘activities’, with cited example categories including Activities, Codes, Dates and Values, Equipment and Instruments, Labels, and Materials), but also introduce tags for topics (‘Specific’ classifications). Scientists also occasionally add ‘key-value’ pairs as metadata (only slightly over half of all notebooks studied used at least one key and only about a third used more than three keys), again dominated by hierarchical classification. Scientists themselves therefore show little imagination in their use of metadata, only appending further cataloging and classification terms.

Additional ways to improve use of record keeping and data storage in scientific and manufacturing advances are needed. Furthermore, additional ways to classify and describe data, for example via use of additional types and/or classes of metadata, would likewise make scientific processes and record keeping thereof more robust.

### BRIEF SUMMARY OF THE INVENTION

The present invention is related to record keeping, data entry, and data file types, and storage methods within electronic and/or empirical data management systems (EDMSs). Collectively, empirical data management systems (EDMS) comprises Laboratory Information Management System (LIMS), Scientific Data Management System (SDMS), Electronic Laboratory Notebook (ELN), and the like.

In one embodiment the invention addresses splicing data about environmental conditions into metadata associated with measurement data received from another device. In another embodiment the present invention provides an aggregated data file having both environmental data together with measurement data. In another embodiment the present invention facilitates access to environmental data; in another embodiment this is used to estimate an offset in actual measurement from a specified measurement that is associated with those environmental conditions; in a further embodiment an estimated actual measurement is provided; and in another embodiment a specified measurement is changed in response to the environmental conditions in anticipation that the actual measurement will fall closer to a desired measurement than it would if no response were made for environmental conditions. When local environmental conditions are provided as contextual information about experimental, manufacturing and/or measurement etc. this provide a rich contextual understanding of the process (laboratory or manufacturing etc.) and thus can provide better understandings as to why, or why not, the experiment worked and/or provide serendipitous discoveries and/or understandings of unexpected results, etc.

In a first preferred embodiment, the present invention provides an electronic laboratory notebook (ELN) system. The ELN comprises an application server running an ELN server application, a data storage system containing data in communication with the application server, and an environmental sensor unit in communication with the application server. The data comprises environmental data received from the environmental sensor unit. In another embodiment, the present invention provides a method of using an ELN having environmental data stored therein. The method includes the step of providing an ELN system as described herein and saving environmental data from an environmental sensor unit in the data storage system.

In another preferred embodiment, the present invention provides an aggregated data file. The file comprises measurement data received from a measurement instrument selected from the group consisting of laboratory equipment and manufacturing facility equipment and environmental sensor data received from an environmental sensor unit and obtained within a time frame of when the measurement data was measured by or received from the measurement instrument.

In a further preferred embodiment, the present invention provides an aggregated data system. The system includes a measurement instrument selected from the group consisting of laboratory equipment and manufacturing facility equipment; an environmental sensor unit; a data aggregation module programmed with logic to receive and aggregate data from the instrument and the environmental sensor into an aggregated data file; and an interface module programmed with logic to transfer the aggregated data file to an external data storage device.

### BRIEF DESCRIPTION OF THE DRAWING

FIG. 1 shows an exemplary file structure in accordance with an embodiment of the present invention.

FIG. 2 shows another exemplary file structure in accordance with an embodiment of the present invention.

FIG. 3 shows experimental data demonstrating how environmental conditions can affect volume dispensed by a pipetting robot.

FIG. 4 shows experimental data demonstrating weight change in caffeine samples at various humidity levels.

FIGS. 5 to 8 show exemplary systems including those in support of electronic laboratory notebook (ELN) and data storage systems according to embodiments of the present invention.

FIG. 9 shows correlation information that can be used in a data analysis step or module.

FIG. 10 shows additional correlation information that can be used in a data analysis step or module.

FIG. 11 show an exemplary laboratory/experiment setup which employs an environmental sensor unit in connection with a network running an ELN.

### DETAILED DESCRIPTION OF THE INVENTION

The present invention provides improvements in record keeping and data storage in scientific and manufacturing processes. The present invention is also related to record keeping, data entry, and data file types, and storage methods within electronic and/or empirical data management systems (EDMSs). Collectively, empirical data management systems (EDMS) comprises Laboratory Information Management System (LIMS), Scientific Data Management System (SDMS), Electronic Laboratory Notebook (ELN), and the like. In some preferred embodiments, ELNs are selected as the EDMS due to the robustness of ELN systems and their capabilities.

Furthermore, the present invention provides additional ways to classify and describe data from laboratory and manufacturing equipment/instruments, for example via use of additional types and/or classes of metadata that make scientific processes, record keeping, and data analysis more robust. Measurement, recordation and use of this additional type/class of metadata can provide higher visibility of process mechanics and process steps which in turn can lead to significant advances in understanding of these processes and their results.

One type of information not represented in metadata previously created and/or recorded in the art is data related to measurements of environmental conditions about instruments (e.g. in a lab or manufacturing facility etc.) at the time measurements are made by these instruments and/or about materials at or around the time these materials are used or stored. This omission reflects the present way that metadata is appended to measurements, accumulating as data passes through a network. Since measurements of environmental conditions are made by sensors that are either peripheral to a network or present only on a separate network or remote sensor, instrument measurements do not cross paths with environmental data and so environmental data does not get ‘stuck’ onto (e.g. appended to) instrument measurements as metadata or some other data file. Furthermore, even though users have the facility to seek out and append such information, the evidence is that they do not do this. It is not clear whether this is because users do not recognize they have the facility to do this, users do not have the skills to use the facilities to do this, or do not think there is any value in doing this. Whatever the reason, there is ample evidence that environmental conditions frequently have important effects on instrument measurements, even if they are overlooked.

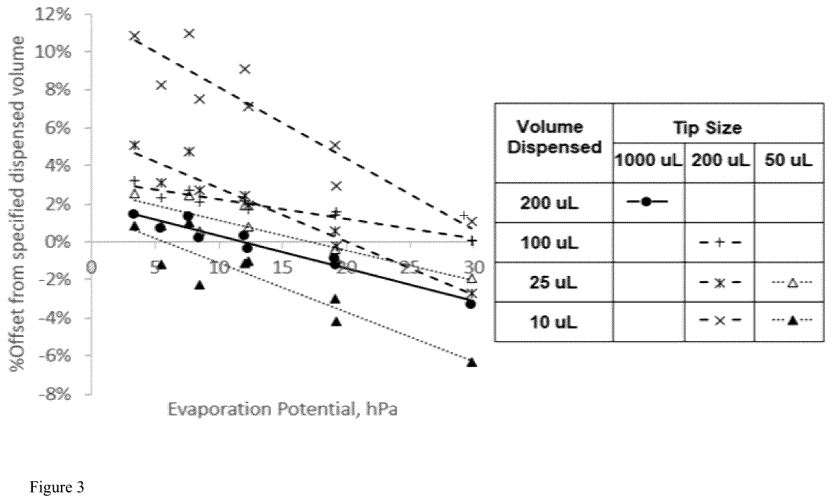

One example of the impact of environmental conditions on instruments is shown in FIG. 3. This shows experimental data from http://www.artel-usa.com/resource-library/does-weather-affect-pipetting-yes/illustrating how environmental conditions can affect volume dispensed by a Tecan Freedom EVO pipetting robot. FIG. 3 shows that variations of 6 to 10% in dispensed volume is possible, according to changes in environmental conditions (in this study, relative humidity of 30 to 80% and temperature of 15 to 30° C.). Although the results have been obtained from only one type of dispenser, it is an aspirate-dispense pipettor using standard pipette tips, so the results are transferrable to a wide range of robotic pipettors and manually operated pipettes.

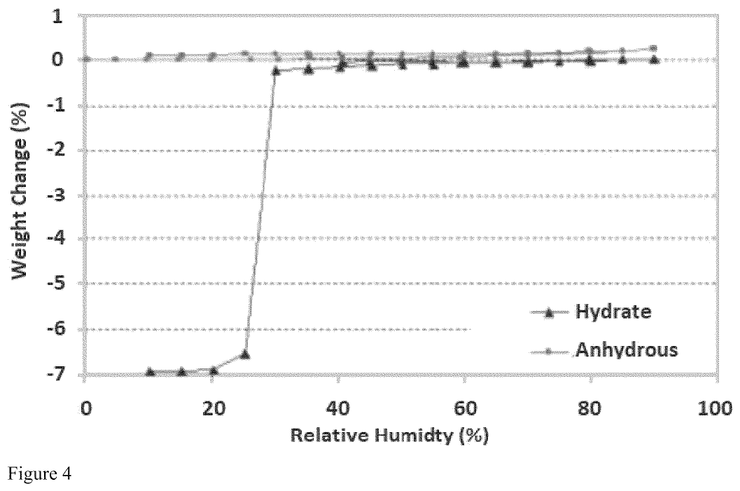

A second example showing the impact of humidity on weighing is shown in FIG. 4. This shows experimental data from ‘Identification of Phase Boundaries in Anhydrate/Hydrate Systems’ J. F. Krzyzaniak G. R. Williams, N. Ni J. Pharma. Sci., 2007 Vol 96, pp 1270-1281. Data shown is weight change in caffeine samples in a VTI moisture balance at 25° C. This shows that where a solid has more than one hydration state, the form that is stable can change according to environmental humidity, having significant impact on the moles of chemical weighed in a specified mass. The paper also shows the transition point between phases changes according temperature. Such impact is not limited to caffeine; many compounds have more than one isolable hydration state—this is sometimes utilized to advantage in well studied cases, e.g. in the color-changing cobalt chloride used in desiccant pellets, but more often is a confounding variable in preparing solutions and reacting compounds in laboratories. Furthermore, change in hydration state is just one reason for moisture absorption; other reasons include pore condensation in fine powders, hygroscopic behavior and reactivity towards water vapor, all of which introduce effects to downstream use of such solids.

Since environmental conditions like temperature and humidity can have detectable and sometimes strong influences on preparation steps like weighing and making solutions, it is clear they will therefore have influence on experiments and measurements made on solids and solutions too. This influence is also likely to be associated with duration of exposure to an environment. However, researchers' ability to identify and react to these influences is going to be limited by their access to data on both the effect and the cause. While the effect, changes in the output of an experiment or measurement, may be noted by researchers, the role in these changes of environmental conditions (and duration of exposure to them) will be missed if measurements of them are not available.

The present Inventors have determined that inverting the logic is more important. In particular, the more available the measurements of environmental conditions are made to researchers (and also to blind, automated, correlation-finding tools) the better the chances are that the impact of environmental conditions on measurements will be identified.

The present invention provides systems and methods of great utility which relate environmental data (preferably with measurement data) in a file system. This can be accomplished via various embodiments described herein where environmental data is aggregated with or appended to measurement data (preferably as metadata) in a file system (e.g. such as one having optical and/or electronic storage means in a file structure and/or file hierarchy etc.).

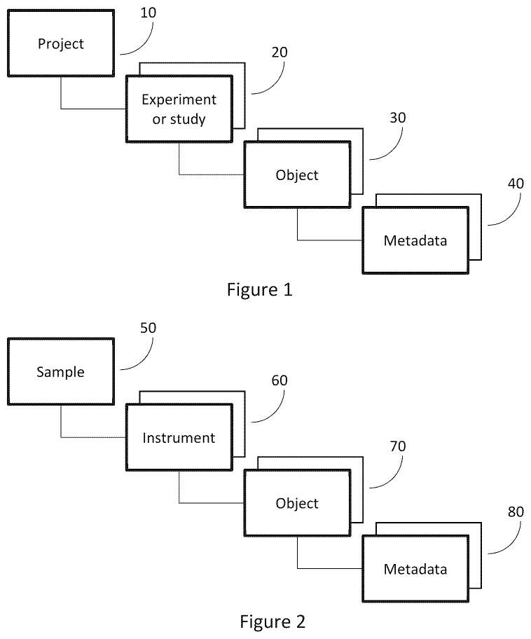

FIGS. 1 and 2 show example file structures of different data types and their dependency provided by an EDMS in accordance with the present invention. In FIG. 1 the file structure follows closely research project management, with a project 10 containing one or more experiments or studies 20, each containing one or more objects 30 such as instrument readings, results, images, graphs etc., each having appended metadata 40. This structure reflects is similar to that described in US patent application 2007/0208800A1 and U.S. Pat. No. 9,489,485. In FIG. 2, a different file structure that follows more closely an analytical laboratory workflow is illustrated, where primary classification is by sample 50, containing one or more Instrument files 60, each containing one or more objects 70 such as instrument readings, results, images, graphs etc., each having appended metadata 80.

FIG. 3 shows experimental data from http://www.artel-usa.com/resource-library/does-weather-affect-pipetting-yes/illustrating how environmental conditions can affect volume dispensed by a Tecan Freedom EVO pipetting robot. The y-axis shows offset in the measurement from the specified dispense volume, calculated as the difference between actual dispensed volume and specified dispense volume, expressed as a % of the specified dispense volume. The x-axis shows Evaporation Potential, which is the shortfall between saturated vapor pressure of water and the actual ambient partial pressure of water. It can be determined as the product of (1−% RH/100)*Pₛₐₜ, where % RH is the relative humidity and Pₛₐₜ is the saturated vapour pressure of water, determined from the measured temperature, T (in Kelvin), using the formula log₁₀(Pₛₐₜ)=−2248.1/T+9.0327.

FIG. 4 shows experimental data from ‘Identification of Phase Boundaries in Anhydrate/Hydrate Systems’ J. F. Krzyzaniak G. R. Williams, N. Ni J. Pharma. Sci., 2007 Vol 96, pp 1270-1281. Data shown is weight change in caffeine samples in a VTI moisture balance at 25° C.

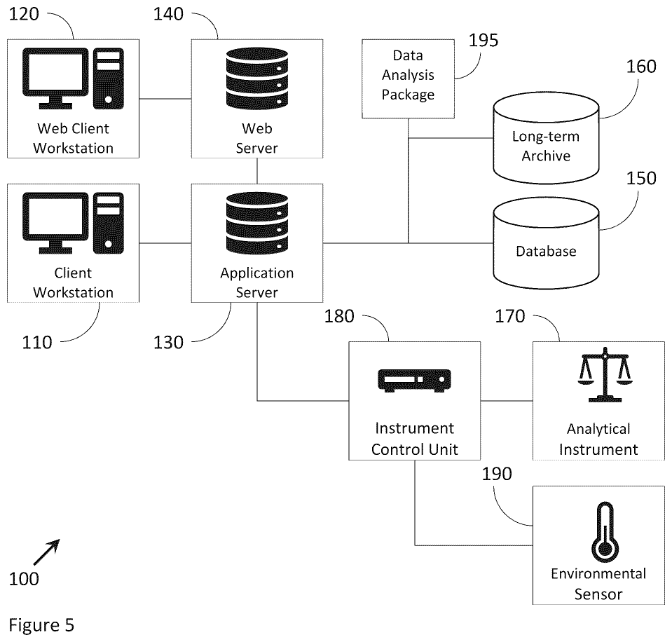

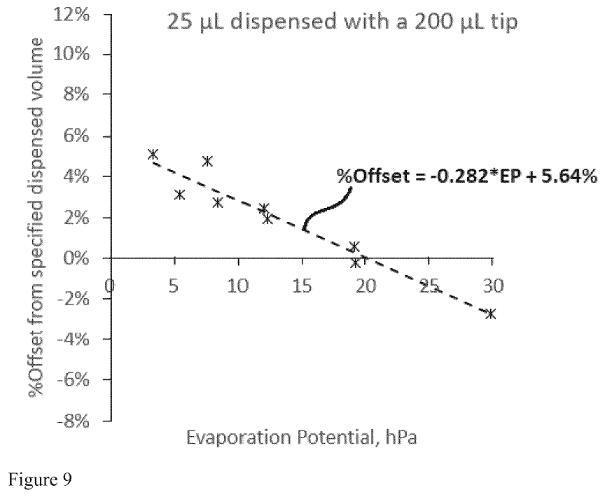

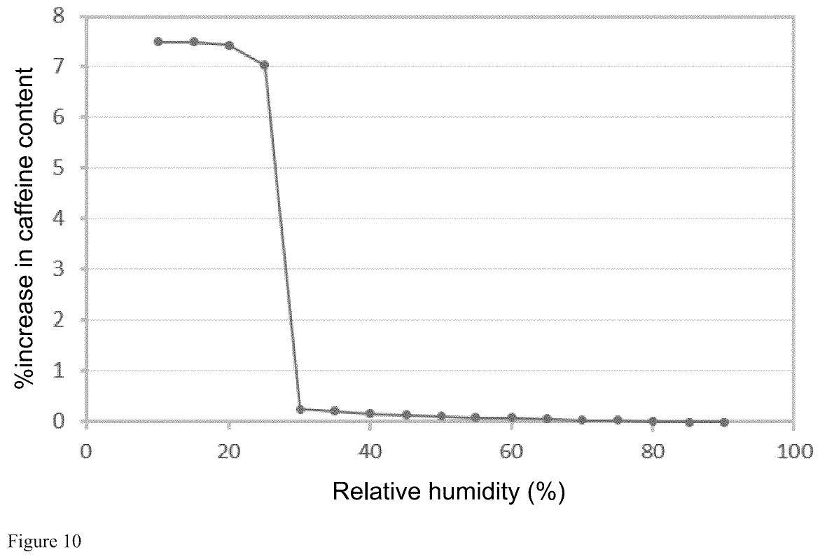

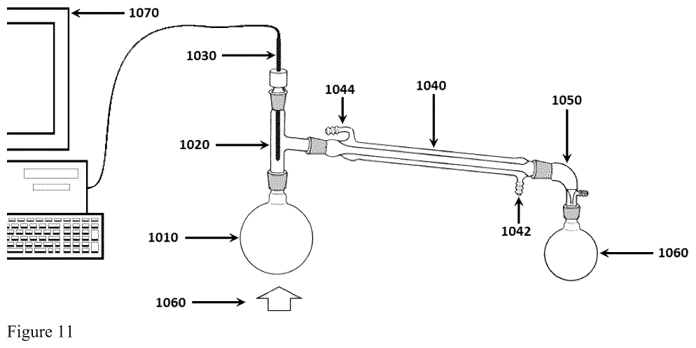

FIGS. 5 to 8 show exemplary systems for supporting the electronic laboratory notebook (ELN) according to the present invention and are more fully described below. FIG. 9 shows correlation information that can be used in a data analysis step or module. FIG. 10 shows additional correlation information that can be used in a data analysis step or module. FIG. 11 show an exemplary laboratory/experiment setup which employs an environmental sensor unit in connection with a network running an ELN.

### Exemplary System Architectures of the Present Invention:

FIG. 5 illustrates an embodiment of an exemplary system 100 for use as, or for supporting, the electronic laboratory notebook (ELN) and/or aggregated data file systems according to the present invention. Two exemplary client workstations 110, 120 are shown which may be connected to the application server 130 using any of a variety of methods known in the art. In this exemplary embodiment workstation 110 is running a full EDMS (e.g. an ELN) application (e.g. a full client workstation), and remote workstation 120 is running a world wide web EDMS (e.g. an ELN) application (e.g. a web client workstation) optionally at on offsite location.

The web client workstation 120 can be connected via the Internet, or alternatively by a web server 140 to a distributed communication network or LAN comprising the application server 130 and optionally the full client workstation 110. It will be recognized that the web client work station 120 also could be directly connected to the LAN. The LAN further includes a shared data storage system or facility 150 (e.g. database 150) and optionally a long-term data storage system or facility 160 (e.g. archive 160). Preferably, the shared database 150 is a multi-user, multi-view relational database such as for non-limiting example an ORACLE database, etc. The long-term data archive 160 is used to provide virtually unlimited amounts of “virtual” disk space (e.g. by means of a multi-layer hierarchical storage management system). The measurement instrument (e.g. analytical instrument 170 or instrument selected from the group consisting of laboratory equipment and manufacturing equipment) is connected to the LAN (an hence to the application server 130) optionally through an instrument control unit 180 and environmental sensor 190 can also be connected to the LAN through instrument control unit 180. One or more data analysis packages/modules 195 may also be attached to the network and or application server. The data analysis packages/modules are programmed with logic/instructions for performing actions on received data such as analyzation, organization, aggregation, sorting, storing, altering, modifying, etc. The present invention is not limited to the illustrated embodiment and more or fewer and equivalent types of components can be used also as would be appreciated by those of ordinary skill in the art.

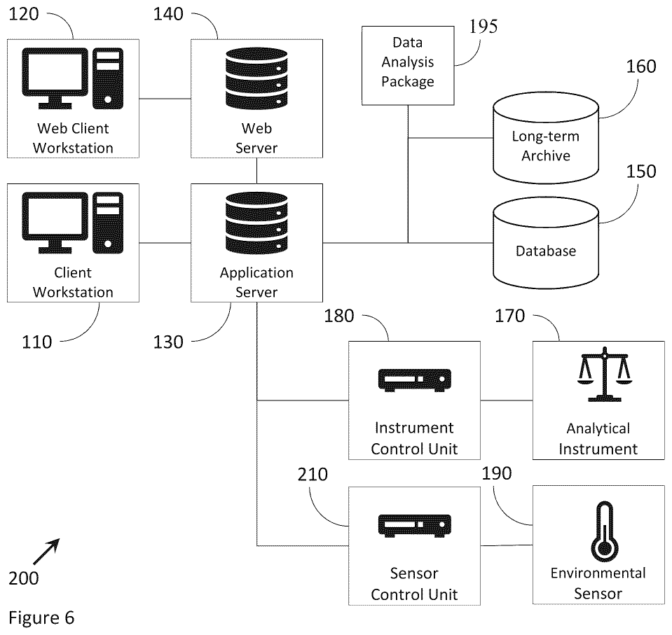

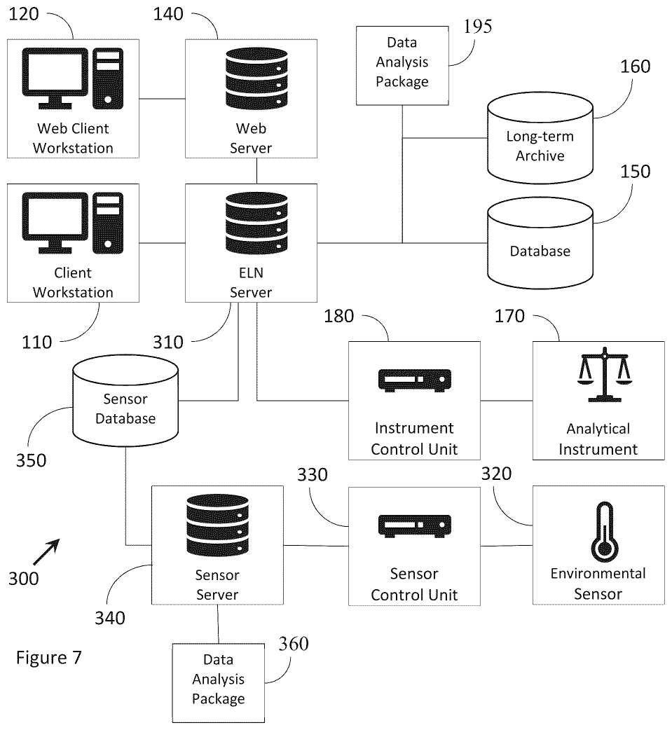

FIG. 6 shows a different system topology 200, where one or more environmental sensors 190 are connected to the Application Server 130 optionally through one or more separate sensor control units 210. FIG. 7 shows an alternative system topology 300, where an EDMS, here an ELN, is supported on an EDMS server, here ELN server 310 but there is a separate sensor network with one or more environmental sensors 320 optionally connected to one or more sensor control units 330 and a Sensor Server 340, with sensor data being stored on Sensor Database 350, to which ELN server 310 has access. The Sensor Server 340 may optionally also have one or more data analysis packages 350. Such a topology may be found for example when environmental sensing and the ELN are provided by separate services.

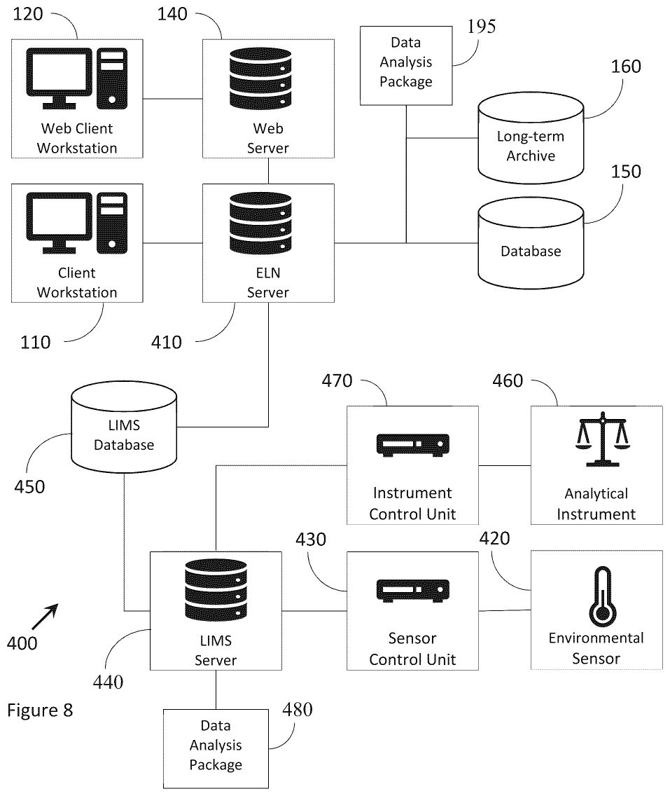

FIG. 8 shows an alternative system topology 400, where the EDMS, here also an ELN, is supported on EDMS server, here ELN server 410 but there is a separate network of a full LIMS with one or more environmental sensors 420 optionally connected to one or more sensor control units 430 and a LIMS Server 440, to which is also connected one or more analytical instruments 460 optionally through an instrument control unit 470, and one or more data analysis packages 480, with sensor data being stored on LIMS Database 450, to which ELN server 310 has access. Such a topology may be found for example when environmental sensing and instrument data management are run by a LIMS service separate from the ELN service.

The various components of the example systems 100, 200, 300 and 400 described above (e.g. the client workstations 110, 120, the application server 130, the web server 140, and the database 150) are preferably completely separated to allow conformity with laboratory/company preferences, workloads, and infrastructure. This can be achieved by adhering to at least a 3-tier client-server architecture or preferably a web-based thin client. Any suitable device connected to the LAN (e.g. a client workstation or an instrument) should be able to interface via TCP/IP to the application server 130, provided the appropriate client software has been installed and configured thereon. Optionally, multiple application servers can be provided which allow for metadata replication. Preferably, the example systems 100, 200 and 300 allow the support of wireless environments, handheld and Tablet PCs, Offline Clients, access via voice-control and the like.

The architecture of the example systems 100, 200, 300 and 400 readily allow the connection of several such LANs all over the world. This is particularly advantageous for globally operating companies that run several research laboratories in different countries and/or continents. Accordingly, all data and related metadata are immediately globally available. Search functions are available for all servers simultaneously. It is possible for a user to access all electronic notebook pages on client hardware anywhere in the world. A support of corporate wide multi-site multi-server storage is, thus, also possible.

In accordance with the embodiments herein described, it can be seen that an EDMS (e.g. electronic laboratory notebook (ELN) system), and/or aggregated data systems, can include an application server running an EDMS and/or ELN server application, a data storage system containing data in communication with the application server, and an environmental sensor unit in communication with the application server. The data comprises environmental data received from the environmental sensor unit.

In preferred embodiments, the EDMS (e.g. ELN) and/or aggregated data systems further include a measurement instrument. In such embodiments, the data storage in the database or storage facility preferably further comprises measurement data received from the measurement instrument. The measurement instrument is not particularly limited and may be selected from the group consisting of any types of laboratory equipment and manufacturing facility equipment.

The data can also comprise the data types selected from the group consisting of project data, experiment data, object data, and metadata. In preferred embodiments the environmental data is saved as metadata.

The environmental sensor unit is not particularly limited. In preferred embodiments the sensor unit is coupled with or in communication with a sensor control unit which either or both are programmed with logic or instructions to receive and/or transfer sensor data to the application service and/or data storage device. In preferred embodiments, the environmental sensor unit measures environmental data selected from the group consisting of temperature, humidity, light intensity, light wavelengths, vibration, gas concentration, air pressure, volatile organic compounds (VOC) concentration, particulate level, and air pollution level.

In preferred embodiments, the systems further include a client workstation running an EDMS (e.g. an ELN) client application in communication with the application server. The data received from the environmental sensor unit is environmental data relating to an environmental condition of the measurement instrument at or about the time measurement data is measured by the instrument and/or transferred to the application server. The environmental data received from the environmental sensor and the measurement date are stored in the data storage system. The environmental data is stored as metadata which characterizes the measurement data.

The measurement instrument is preferably controlled by a controlling computer or module programed with logic and/or instructions for such control. For example, a measurement instrument agent module can being run on the controlling computer, wherein the measurement instrument agent module is programmed with logic to transfer measurement data from the measurement instrument to the application server. In additional embodiments, the EDMS (e.g. ELN system) can includes an instrument interfacing module programmed with logic and/or instructions for establishing a controlled flow of data between the application server and the measurement instrument and/or the environmental sensor unit.

The EDMS (e.g. ELN) and/or aggregated data systems can further comprise a correlation module (e.g. optionally resident or coextensive with the data analysis packages 195 of FIGS. 5-8 programmed with logic and/or instructions to determine if a correlation exists between the measurement data and environmental data. In these embodiments, correlation determination can be performed via statistical analysis and/or statistical comparison of the measurement data and the environmental data. In such embodiments, if a correlation is determined the correlation module and/or application server is programmed with logic and/or instructions to perform or suggest performance of an action and/or step. Such action and/or step is preferably selected from the group consisting of: (i) modifying the measurement data; (ii) calculating a correction or offset factor for the measurement data; (iii) modifying a result: (iv) generating an informational, error, and/or warning message to send to or display to a user; (v) modifying or a process step process run, or process protocol; (vi) mathematically modeling the identified correlation (e.g. via mathematical relationship, plotting, three dimensional vectors, multi-dimensional arrays, or tensor), (vii) terminating a process step, process run, or process protocol; and (viii) saving in the EDMS (e.g. ELN system) or aggregated data file (preferably as additional metadata) OR displaying on a display (preferably a client or web-client work station) information related to any of (i) to (vii) in the data storage system or on a display.

In the embodiments described herein, the present invention provides an EDMS (e.g. ELN system) and/or aggregated data system and/or aggregated data file containing measurement data received from measurement equipment and environmental data received from an environmental sensor. In preferred embodiments, the environmental data describes environmental data about said measurement equipment at about the time of measurement data is obtained. In further preferred embodiments, the environmental data is saved as metadata (optionally in an aggregated data file) with said measurement data.

### Environmental Sensor Data and Measurement Instrument Data Collection and Aggregation/Appending:

In the embodiments herein described, the EDMS (e.g. ELN system) and/or aggregated data systems (and methods of use etc.) make use of computer infrastructure/modules programmed with logic/instructions and having circuitry comprised of hardware, software, memory, processors, data storage, computers, etc. which cause/create/effect operability of said systems and methods.

The present invention also provides a method of appending environmental measurements as metadata to instrument measurements. In the context of system architecture, there are many ways to append environmental data as metadata. Preferred examples of these include, for example:
An instrument control unit such as 180 in System 100 of FIG. 5, can collect data directly from Environmental sensor 190 and add it as metadata along with other measurement metadata
An instrument Control Unit such as 180 in System 200 of FIG. 6 can collect and aggregate data from
   Sensor control unit 210
   Application server 130
   Database 150
An Application server such as 130 in System 200 of FIG. 6 can collect environmental data from optional sensor control unit 210 or directly from environmental sensor 190 and append it as metadata to measurement data and metadata from instrument control unit 180
An EDMS (e.g. ELN system) server such as 310 in System 300 of FIG. 7 can collect environmental data from sensor database 350 and append it as metadata to measurement data and metadata from instrument control unit 180
A LIMS server such as 440 in System 400 of FIG. 8 can collect environmental data from optional sensor control unit 430 or directly from environmental sensor 420 and append it as metadata to measurement data and metadata from instrument control unit 470
Etc.

In the context of identifying environmental measurement from data streams of environmental sensors, the following are commonly useful:
Time point of measurement; appropriate for simple instrument measurements like balance weights
Time point immediately before disturbance prior to measurement; appropriate for e.g. storage conditions of substances.
Two time points of measurements: one at start of measurement, one at end. In this case, duration data (i.e. difference between start time and end time) is also valuable metadata.
Statistical summary across duration of measurement (mean, stdev)

Several different environmental factors can be measured using the various embodiments described herein. The word ‘Environment’ can be for example: the area where an instrument (lab or manufacturing equipment where measurement or other related data is obtained from); a laboratory or part of a laboratory space, a cold room, an animal house, a manufacturing floor, a greenhouse, a weather station; the area surrounding a chemical or ingredient being measured, or involved in the preparation of samples being measured, such as a reagent bottle (as measured by a miniaturized sensor or array of sensors, a ‘smart lid’ etc.), any storage container (grain silo, fermentation tank, refrigerator, freezer, etc.).

The environmental factors (e.g. measured environmental parameters) can be, for example any of the following: temperature, humidity, atmospheric pressure, gas composition (overall, or specific to certain components of interest such as Volatile Organic Compounds (VOCs), ammonia, carbon monoxide, carbon dioxide, oxygen, or any other molecule for which sensors are available) light intensity (overall, or specific to a window of wavelengths-red, green, blue, or otherwise filtered to be sensitive only to a range of frequencies useful to the application, such as blue-UV for light-sensitive chemistry, or near infra-red, red and blue for plant growth) sound intensity (overall, or specific to a window of frequencies), motion, changes in magnetic strength or orientation etc.

Another environment factor related to the instrument measurement data that can be measured by environmental sensing units is “whom took the measurement” and/or the “Time of measurement” from the laboratory/manufacturing facility equipment or “duration of a process step”. Such a measured factor can give a measure of the environment representative of conditions such as when using the instrument and/or inside a reagent container immediately before use. Further such a measured factor can give duration data (i.e. difference between times of measurements of other process steps) and this can also be determined from measured and recorded time points. This factor can be determined by any known methods of determining time or duration of time. In the alternative this factor can be determined by: a change of state in measuring equipment (e.g. change in weight recorded by a balance, motion detected by motion sensor (such as an accelerometer, gyroscope, software-based gyroscope) fitted to portable equipment or reagent containers etc.). In the alternative it simply can be determined and input by the operator of the equipment.

The choice of what environmental factor(s) to measure can be guided by relevance to the measurement (known or suspected by instrument manufacturer, research and supervisory staff) and availability of sensors (both commercially and the subset installed by an institution). The location of sensors needs to be adequate to represent the local environment but this may not mean close spatially; for example, atmospheric pressure across an entire floor of a building may be equal if there are no positive-pressure areas like clean rooms or negative-pressure areas like biohazard containment areas, and so an atmospheric pressure sensor somewhere on that floor can often be used to supply environmental pressure data relevant to the entire floor. In contrast, storage humidity may require a far more local sensor within a reagent container. Handling humidity may be recorded by a nearby humidity environmental sensor, but if there are no sources of water vapour addition (humidifiers, hot water baths etc.) or extraction (dehumidifiers, areas of water condensation) a more remote humidity sensor can be used; however, relative humidity varies with temperature and so corrections may be needed for temperature differences, using dew point or water vapour pressure as a constant point for correction.

US Prov. Application entitled “Method and Apparatus for Local Sensing” which was filed on Oct. 1, 2018 and received U.S. Provisional Application Ser. No. 62/739,419 (which is incorporated herein by reference) describes a label/tag sensor package comprising a plurality of sensors configured on a small flexible backing for local sensing applications. This smart label sensor package can be placed on laboratory/manufacturing equipment, storage containers, and even on products and/or packaging as the product is produced, stored and/or shipped. This sensor package can measure/determine many of the environmental factors of interest and described herein and can wirelessly communicate this data to an application server for aggregating with measurement data received from process instruments in the methods herein described. Furthermore, due to the size and relatively low cost of these sensor packages, they can be placed at many different locations (e.g. such as on tools and instruments) within a facility and measure local environmental conditions with ease, etc.

Methods of Use of a File Hierarchy Containing Environmental Data and Instrument Data (e.g. Environmental Data Saved as Metadata):

The present invention also provides methods of using the ELNs and/or aggregated data files and systems described herein which have environmental data aggregated with and/or appended to (preferably as metadata) equipment/instrument measurement data.

In one embodiment simply having access to environmental data is of extreme benefit to users. In other words, having access to environmental data on a client workstation and/or web client workstation allows for higher visibility of the process and its results. It allows for inspection by researchers in an EDMS (e.g. ELN system), where the EDMS (e.g. ELN system) supports display of metadata by hovering over the measurement. While this gives only on-screen, visual access to the environmental conditions, it allows researchers (or data analysis packages 195 of FIGS. 4-8, etc.) to do rapid screening of possible correlations between measurements, outcomes and environmental factors or validation that protocols were executed within specified limits. Such screening and validation activities of environmental conditions will therefore be executed more quickly and efficiently when environmental data is aggregated with and/or other stored with measurement data as for example metadata.

Having access to environmental data on a client work station and/or web client work station also facilitates data analysis by researchers, where metadata is downloaded with requested data in a format suitable for use in spreadsheets (.csv .txt, proprietary e.g. .xlsx .gsheet etc.). This allows researchers to work with data on their preferred platform to search for correlations; optionally, evidence of such correlations can then be posted in the ELN. For example, correlations may be linear or non-linear trends in data; and/or identification of specific conditions or combinations of conditions that lead to unfavorable outcomes.

Having access to environmental data coupled with equipment/instrument measurement data from the process also allows for improved automated analysis. FIGS. 5 to 8 show example system architectures that can support the invention; in all of these, optional data analysis modules/packages (programmed with data analysis logic and/or instructions) are shown (195, 360, 480) as part of the architecture. There are many configurations of systems that allow data to be analyzed by such packages or equivalent and there are many ways in which data can be analyzed, but an example form is correlative analytics, where data is searched to identify measurements of one or more parameters that correlate with measurements of other parameters. Correlation is often identified by statistical testing, but when a correlation search is used as a blind tool across a family of data sets, statisticians recognize its power is diminished because of the need to avoid increasing the likelihood of false discoveries; this topic is the ‘Familywise Error Rate’ (FWER) and approaches to manage its impact include the Bonferroni procedure, The Šidák procedure (see “Rectangular Confidence Regions for the Means of Multivariate Normal Distributions” by Z. K. Šidák, Journal of the American Statistical Association 1967 Vol 62 pp 626-633) and more recent approaches such as that described in ‘Controlling the False Discovery Rate: a Practical and Powerful Approach to Multiple Testing’ by Y Benjamini and Y Hochberg J. Royal Statistical Soc. B 1995 Vol 57 pp 289-300. It is therefore preferable to limit correlation searches to small families of relevant data sets where possible, rather than an entire database, to avoid diluting their power. A simple way to do this is to limit the family of data sets to those that are closely related; metadata is therefore a valuable resource for correlation searches, and inclusion of environmental data in the metadata is justified by the examples where it has been found previously to be a factor in experiment outcomes.

US Provisional Applications both of which are entitled “Method and Apparatus for Process Optimization” which were filed on Oct. 1, 2018 and Feb. 4, 2019 and which received U.S. Provisional Application Ser. Nos. 62/739,441 and 62/800,900 which are incorporated herein by reference, describe methods for determining whether processes are on a trajectory for successful completion by observing and/or correlating environmental data observed/measured in a current run with environmental data observed/measured during previous runs of the process. If it is determined that the process is not of a trajectory for success the process may be abandoned, or the protocol may be altered such that the given run is put back on a course/trajectory for successful completion. Logic and/or instructions for such analysis of data may be incorporated into the data analysis packages herein described.

In another embodiment, analysis of a file system containing environmental condition data can also facilitate equipment maintenance and/or determining maintenance schedules in the laboratory and/or manufacturing facility. Logic and/or instructions for such analysis of data may be incorporated into the data analysis packages herein described. The following scenario is exemplary of this embodiment:
An example piece of equipment is a freezer, which may be fitted with a switch to detect door-opening events. Example devices for detecting door-opening events include a latching switch (U.S. Pat. No. 3,996,434); a magnetic switch (U.S. Pat. No. 4,241,337); a capacitive sensing switch (U.S. Pat. No. 4,691,195); and a light-detecting indicator coupled to a fridge or freezer light.
An example maintenance cycle is a freezer defrosting cycle and, since frosting up of freezer is caused by condensation of water vapour from warm, moist air that enters the freezer, principally when the door is opened, timing of the freezer defrosting cycle can be improved by considering door opening events. Prior art in U.S. Pat. No. 4,463,348 discloses that freezer defrosting can be tied to a simple cumulative time the door is detected to be open.
Freezer maintenance can be refined beyond what is possible using simple time data for freezer door-opening events, since simple time data will only indicate how much air exchange may occur but not how much moisture that air carries and hence how much frost may form in the freezer. However, if the humidity of the environment outside the freezer is measured and appended to the time data, it can be considered by an algorithm that predicts when a freezer may be losing efficiency due to accumulated frost to improve prediction of when the next defrost cycle is due.
Other equipment where exposure to moisture during use is a concern will also benefit from a maintenance schedule that can be tailored by a scheduler with ready access to humidity data associated with use.

As described herein analysis of a file system having environmental condition data and instrument measurement data can be used to identify correlations between these different data sets. Furthermore, the present invention provides methods using these identified correlations to improve the underlying process such as in estimating, calculating or otherwise determining alternative/improved results and/or correction factors for altering or improving instrument measurements. In some embodiments modifications are made to the measurement data, to the actual process protocol, or to the results achieved by the process. The following scenarios are exemplary of these concepts and use of identified correlations between environmental conditions and instrument measurements.
Where a correlation between a measurement and environmental conditions has been established, it becomes possible to use this correlation to enhance the metadata.
   For example, for the Tecan Freedom EVO pipetting robot used in FIG. 3, fitted with a 200 μL tip and set to dispense 25 μL, a correlation between evaporation potential and % offset from specified dispense volume has been identified as illustrated in FIG. 9. Once this correlation is known, it can be used to estimate a % offset, calculated from the environmental conditions. For a particular measurement of 25 μL, when the relative humidity is measured to be 10% RH and the temperature is 25° C., such as is common in a heated New England laboratory during winter, the Evaporation Potential is 27.97 hPa (with an intermediate calculation of Psat=31.084 hPa); as an input to the trend shown in FIG. 9, this gives an offset of −2.25% in dispensed volume. Under the conditions of a New England summer, where laboratory humidity can increase to e.g. 50% but lab temperature is maintained at 25° C. (so that again Psat=31.084 hPa), the evaporation potential will fall to 15.54 hPa and the offset becomes +1.26%. The metadata for a dispense can be enhanced considerably by inclusion of the estimated offset, and the intermediate calculations of Psat and Evaporation potential can potentially also be included.
   Another example is for weighing caffeine hydrate, which is recognized to dehydrate when handled below 30% RH, as illustrated in FIG. 4. This can be rearranged to determine the % increase in caffeine content, as shown in FIG. 10. This shows that at humidities below 30% RH, there is a marked increase in caffeine content in the weighed amount, which can be estimated to be +7.5%. This new rule can be applied in estimating the effect of room-temperature humidity on weighing caffeine hydrate; for laboratory conditions of 10% RH and 25° C., such as is common in a heated New England laboratory during winter, the caffeine hydrate will dehydrate, resulting in an estimated offset of +7.5%, while in a New England summer at 50% the offset in weight can be estimated as 0%. The rule can be adapted to improve estimates, e.g. by spline fitting or curve fitting the data, without changing the underlying method.
in a further embodiment an estimated actual measurement is provided:
   Extending the previous examples of the Tecan Freedom EVO pipetting robot fitted with a 200 μL tip and set to dispense 25 μL, when the relative humidity is measured to be 10% RH and the temperature is 25° C., such as is common in a heated New England laboratory during winter, the offset has been estimated to be −2.25%, so an estimate of actual dispensed volume of 23.4375 μL can be added to the metadata. Under the conditions of a New England summer, where laboratory humidity can increase to e.g. 50% but lab temperature is maintained at 25° C., the offset has been estimated to be +1.26%, so an estimate of actual dispensed volume of 25.315 μL can be added to the metadata.
   Another example concerns distillations of liquids. In a laboratory set-up where a liquid is distilled, a temperature probe measuring the temperature of the vapor above the boiling liquid can replace the usual glass thermometer to become the analytical instrument 170 of FIG. 5, 6, or 7 or 460 of FIG. 8. The instrument control unit 180 or 470 performs the function of identifying plateaus in the vapor temperature to generate boiling points. An example set-up is illustrated in FIG. 11 although chemists ordinarily skilled in the art will recognize that apparatus for distillation can be set up in many different ways. FIG. 11 shows a distilling flask 1010 connected to a three-way adaptor 1020 equipped with a temperature probe 1030 and connected to a condenser 1040 with water inlet 1042 and water outlet 1044, connected to receiving flask 1060 via connector 1050. A heat source 1060 for supplying heat to the contents of distilling flask 1010 is also supplied, as is an analytical instrument 1070 for collecting and analyzing data from the temperature probe 1030. This connection is illustrated as a physical cable, but in other embodiments can also be wirelessly connected.
   Environmental data that includes atmospheric pressure can be collected by an environmental sensor 190 of FIG. 5 or 6, 320 of FIG. 7, or 420 of FIG. 8; atmospheric pressure data can in one embodiment be incorporated into the metadata. In another embodiment, repeats of the same distillation procedure can generate a set of distillation temperatures and pressures, which can be fitted by an appropriate model such as the Clausius-Clapeyron equation:

$$ln ( \frac{{P}_{1}}{{P}_{2}} ) = \frac{Δ {H}_{v a p}}{R} ( \frac{1}{{T}_{2}} - \frac{1}{{T}_{1}} )$$

In a further embodiment, Trouton's rule, ΔSᵥₐₚ≈10.5R, can be used to fix the intercept implied by the Clausius Clapeyron equation:

$$ln \frac{P}{{P}^{θ}} = - \frac{Δ G}{R T} = \frac{Δ {S}_{v a p}}{R} - \frac{Δ {H}_{v a p}}{R T} \approx 10.5 - \frac{Δ {H}_{v a p}}{R T}$$

Note that during a distillation, the pressure of the distillate will equal atmospheric pressure, P, at the distillation plateau temperature T; since standard pressure, P^{Θ}, is known (1013.25 hPa), this allows estimates of the enthalpy of evaporation ΔHᵥₐₚ to be made from a single distillation and then used to determine a distillation temperature at another pressure, such as at standard atmospheric pressure. Either or both can be usefully incorporated into the metadata.
   This illustrates that modelling correlations between measurements and environmental conditions does not need to be based solely on empirical fitting of data; it can also fit data to known relationships to generate other metadata.
In another embodiment a specified measurement is changed in response to the environmental conditions in anticipation that the actual measurement will fall closer to a desired measurement than it would if no response were made for environmental conditions.
   The utility of this embodiment is best realized in a pre-defined procedure. As noted in the Background section, an EDMS (e.g. ELN system) can provide both a flexible platform to support research work and more structured interfaces tailored to particular tasks, and it is the more structured interface that is used to support record keeping for pre-defined procedures.
   Predefined procedures may or may not have an existing body of data supporting choice of steps. Therefore, relationships between environmental conditions and specific steps of the protocol, such as the impact of humidity on weighing of substances, the impact of temperature and humidity on pipetting of volumes, the impact of atmospheric pressure on distilling etc., may or may not already have been elucidated or may have been estimated from limited data and application of known relationships, insight and/or experience. Any of these types of recognized relationships can be applied in this embodiment and any can be improved or replaced in light of further evidence.
      Definition of relationships and/or improvement of recognized relationships can be made at the time of establishing a protocol or any time thereafter. Modifications to an existing protocol in light of newly established or improved definitions may be made manually, may be managed through a quality system with review of evidence and sign-off, or may be made automatically if a correlation identified by an automated analysis package reaches a predefined level of confidence.
   A specified measurement may be changed in response to altered environmental conditions in a variety of ways:
      The determined relationship may be applied to environmental conditions that lie within the range of those previously experienced (i.e. interpolation) as well as conditions beyond those already experienced (i.e. extrapolation)
      A limit of environmental conditions may be applied so that change is only made under interpolation conditions. Where extrapolation would be required, it is possible to specify no adjustment, or to apply an adjustment no more extreme than one already justified by the limit of known environmental conditions, or some other change to an extrapolation that allows the protocol designer to apply a degree of caution.
      A protocol may be changed in response to environmental conditions by abandoning the protocol where it is recognized that the conditions will not allow for success in executing the protocol. Examples of this condition include a humidity too high for a preparation to be dried successfully or too low for tissue samples to be handled without damage, a temperature too cold for equipment to operate successfully (such as compromised O-rings, lubricants being too viscous, reagents having frozen etc.) or too warm for success (excessive evaporation of solvents, enzyme denaturing, a light level too damaging for photosensitive components or too dim for necessary photocatalysis, vibration levels too high for successful use of an analytical balance etc.
   A protocol may be modified through relationships between environmental data and measurements that have been identified by analysis of metadata; optionally, protocols may also be modified by analysis of measurement data and environmental data not associated in a measurement-metadata relationship, such as data stored in different file locations in disk storage, or on different disks, or in different databases, or in different data management systems.
   An example of changing a specified measurement in response to altered conditions Is given in the context of operating the Tecan Freedom EVO pipetting robot fitted with a 200 μL tip and specified to dispense 25 μL, this specification having be made when operating at 25° C. and 35.7% RH (i.e. an Evaporation potential of 19.99 hPa, where no offset is found between specified volumes and actual dispensed volumes). When the protocol calls for a step to do this, but querying the environmental sensor identifies the humidity to have fallen to 10% RH (while temperature is stable at 25° C.), the dispenser is recognized in the examples above to have an offset of −2.25% in dispensed volume, dispensing only 23.4375 μL. In this case, the protocol updates in response to the environmental sensor readings to specify a volume to dispense of 25.575 μL, because the best estimate of actual dispense volume, after considering the impact of the lower humidity, will be the desired 25.0 μL
   Another example of changing a specified measurement in response to altered conditions is given in the context of transferring a distillation protocol from a lab in Boston to a partner one in Denver. Such sharing of protocols can follow the teaching of e.g. U.S. Pat. No. 9,842,151. For a protocol defining synthesis of trimethyl (phenoxy) silane from iodotrimethylsilane, where product and starting material are separated by distillation. In the Boston lab, a first fraction is collected at 106-109° C. (environmental metadata: P=1014.7 hPa, estimated ΔHᵥₐₚ=33.2 kJmol⁻¹, determined as described in a previous example) and a second fraction is collected at 119-120° C. (environmental metadata: P=1014.7 hPa, estimated ΔHᵥₐₚ=34.3 KJmol⁻¹), the first fraction being unreacted iodotrimethylsilane and the second fraction being the desired product trimethyl (phenoxy) silane. Transfer of these fraction temperatures and associated metadata in a protocol to the Denver lab then means the protocol functioning according to the invention, specifically here applying a pressure correction to the boiling points, has the necessary information in the metadata to update specification for the product boiling range in the protocol. If, for example, the laboratory pressure at time of distillation is measured to be 830 hPa, a first fraction distilling at 98.9-101.8° C. can be identified as unreacted starting material and a second fraction distilling at 111.6-112.6° C. can be identified as product. This uses a rearrangement of equation used to generate values for ΔHᵥₐₚ, specifically:

$$T = \frac{Δ {H}_{v a p}}{R ( 10.5 - ln \frac{P}{{P}^{θ}} )}$$

Changing the specified temperature range reacts to the difference in atmospheric pressure caused by difference in altitude of the two labs, and prevents technical staff running the protocol from mis-identifying the desired product as unreacted starting material.

This application is also related to US Prov. Applications entitled (1) “Method and Apparatus for Local Sensing” which was filed on Oct. 1, 2018 and received U.S. Provisional Application Ser. No. 62/739,419; (2) “Method and Apparatus for Process Optimization” which was filed on Oct. 1, 2018 and received U.S. Provisional Application Ser. No. 62/739,441; and (3) “Method and Apparatus for Process Optimization” which was filed on Feb. 4, 2019 and received U.S. Provisional Application Ser. No. 62/800,900. These provisional applications are incorporated in their entireties herein by reference for all purposes.

The following references are also referred to in this application:
US20070208800 A1
U.S. Pat. No. 6,725,232
U.S. Pat. No. 7,250,950
U.S. Pat. No. 7,555,492
U.S. Pat. No. 8,548,950
U.S. Pat. No. 8,984,083
U.S. Pat. No. 9,489,485
U.S. Pat. No. 9,842,151
U.S. Pat. No. 9,954,976
‘Creating Context for the Experiment Record. User-Defined Metadata: Investigations into Metadata Usage in the LabTrove ELN’ by C. Willoughby, C. L. Bird, S. J. Coles and J. G. Frey in the Journal of Chemical Information and Modeling, 2014, Vol 54 pp 3268-3283.
http://www.artel-usa.com/resource-library/does-weather-affect-pipetting-yes/
‘Identification of Phase Boundaries in Anhydrate/Hydrate Systems’ J. F. Krzyzaniak G. R. Williams, N. Ni J. Pharma. Sci., 2007 Vol 96, pp 1270-1281.
“Rectangular Confidence Regions for the Means of Multivariate Normal Distributions” by Z. K. Šidák, Journal of the American Statistical Association 1967 Vol 62 pp 626-633
‘Controlling the False Discovery Rate: a Practical and Powerful Approach to Multiple Testing’ by Y Benjamini and Y Hochberg J. Royal Statistical Soc. B 1995 Vol 57 pp 289-300.

Any external reference mentioned herein, including for example websites, articles, reference books, textbooks, granted patents, and patent applications are incorporated in their entireties herein by reference for all purposes.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that the described element(s) may be combined in any suitable manner in the various embodiments.

Numerical values in the specification and claims of this application reflect average values for a composition. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

1.-23. (canceled)

24. An empirical data management system (EDMS) comprising:
a server;
a server application comprising an aggregated data file creation module and a correlation module, the server application running on the server;
a measurement instrument in communication with the server, wherein the measurement instrument is a laboratory instrument or a manufacturing facility instrument and is configured to transfer the measurement data to the server;
an environmental sensor in communication with the server, wherein the environmental sensor is configured to determine environmental data about the measurement instrument and transfer the environmental data to the server; wherein the environmental sensor is configured to determine environmental data about the measurement instrument selected from the group consisting of: temperature, humidity, light intensity, light wavelengths, vibration, gas concentration, air pressure, volatile organic compounds (VOC) concentration, particulate level, and air pollution level;
a data storage system in communication with the server; and
an aggregated data file stored in the data storage system, the aggregated data file comprising the measurement data received by the server from the measurement instrument and metadata comprising the environmental data received by the server from the environmental sensor unit, wherein the metadata is appended to and stored with the measurement data in the aggregated data file by the aggregated data file creation module, and wherein the metadata describes the environmental data about the measurement instrument at the time measurement data is measured by the measurement instrument;
wherein:
the aggregated data file creation module is programmed with logic and instructions: to receive measurement data from the measurement instrument and to receive environmental data from the environmental sensor; to create the aggregated data file by appending the environmental data received from the environmental sensor as metadata to the measurement data received from the measurement instrument; and to store the aggregated data file in the data storage system; and
the correlation module is programmed with logic and instructions: to receive the aggregated data file created and stored in the data storage system by the aggregated data file creation module; to determine if a correlation exists between the measurement data and the environmental data stored as metadata within the aggregated data file; and when a correlation is determined to exists to perform or suggest performance of a step selected from the group consisting of: (i) modifying the measurement data stored in the aggregated data file; (ii) calculating a correction or offset factor for the measurement data stored in the aggregated data file; (iii) modifying a result; (iv) generating a display message, an error message, and/or warning message to send to or display to a user; (v) modifying a process step, process run, or process protocol; (vi) mathematically modeling the identified correlation; (vii) terminating a process step, process run, or process protocol; and (viii) saving in the EDMS or an aggregated data file OR displaying on a display information related to any of (i) to (vii) in the data storage system or on a display.

25. The EDMS as recited in claim 24, wherein the measurement instrument and the environmental sensor are individually, or in combination, controlled by one or more controlling computers, wherein the one or more controlling computers associate time stamps with measurement data and environmental data, wherein the aggregated data file creation module creates the aggregated data file by aligning and appending environmental data as metadata to measurement data based upon associated time stamps.

26. The EDMS of claim 24, further comprising:
an instrument control unit in communication with the application server,
wherein the instrument control unit controls the measurement instrument and the environmental sensor unit, and wherein the measurement data received from the measurement instrument and the environmental data received from the environmental sensor unit flow through the instrument control unit to the application server.

27. The EDMS of claim 24, further comprising:
a client workstation running an EDMS client application in communication with the server;
an instrument interfacing module programmed with logic for establishing a controlled flow of data between the server, the measurement instrument, and the environmental sensor unit;
wherein the data storage system further comprises data types received by the server from the client work station and being selected from the group consisting of project data, experiment data, and object data.

28. The EDMS as recited in claim 24, wherein the measurement instrument is controlled by a controlling computer, wherein a measurement instrument agent module runs on the controlling computer, wherein the measurement instrument agent module is programmed with logic to transfer the measurement data from the measurement instrument to the server.

29. The EDMS as recited in claim 24, wherein the EDMS comprises an instrument interfacing module programmed with logic for establishing a controlled flow of data between the application server and the measurement instrument and/or the environmental sensor.

30. The EDMS of claim 24, wherein the data storage system further comprises data types received by the server and being selected from the group consisting of information relating to the identify of a person who used the measurement instrument and the duration of time of the measurement instrument was used.

31. The EDMS of claim 24, wherein the environmental sensor is a humidity sensor configured to measure humidity and/or a temperature sensor configured to measure temperature about the measurement instrument at the time the time a measurement is made, and wherein the aggregated data file stored in the data storage system comprises the measurement data received by the server from the measurement instrument and metadata comprising the temperature and/or humidity about measurement instrument at the time the measurement data was measured by the measurement instrument.

32. The EMD of claim 31, wherein a correlation is determined to exists between the measurement data and the environmental data stored as metadata within the aggregated data file, and wherein the correlation module is programmed with logic and instructions to perform or suggest performance of a step selected from the group consisting of: (i) modifying the measurement data stored in the aggregated data file; (ii) calculating a correction or offset factor for the measurement data stored in the aggregated data file; (iv) generating a display message, an error message, and/or warning message to send to or display to a user; (v) modifying a process step, process run, or process protocol; (vii) terminating a process step, process run, or process protocol; and (viii) saving in the EDMS or an aggregated data file OR displaying on a display information related to any of (i) to (vii) in the data storage system or on a display.

33. The EDMS of claim 31, wherein quality of the measurement data as measured by the measurement instrument is affected by humidity and/or temperature about the measurement instrument at the time the measurement data is measured.

34. The EDMS of claim 24, wherein the environmental sensor is a vibration sensor configured to measure vibration about the measurement instrument at the time the time a measurement is made, and wherein the aggregated data file stored in the data storage system comprises the measurement data received by the server from the measurement instrument and metadata comprising the vibration data about measurement instrument at the time the measurement data was measured by the measurement instrument.

35. The EDMS of claim 34, wherein quality of the measurement data as measured by the measurement instrument is affected by vibrations about the measurement instrument at the time the measurement data is measured.

36. The EDMS of claim 24, wherein the environmental sensor is a light sensor configured to measure light intensity and/or light wavelengths about the measurement instrument at the time the time a measurement is made, and wherein the aggregated data file stored in the data storage system comprises the measurement data received by the server from the measurement instrument and metadata comprising the light intensity and/or light wavelengths about measurement instrument at the time the measurement data was measured by the measurement instrument.

37. The EDMS of claim 36, wherein quality of the measurement data as measured by the measurement instrument is affected by light intensity and/or light wavelengths about the measurement instrument at the time the measurement data is measured.
